(19) United States
(12) Patent Application Publication
(10) Pub. No.: US 2019/0167226 A1
(43) Pub. Date: Jun. 6, 2019

(54) INFANT GASTROINTESTINAL MONITOR

(71) Applicant: INTERNATIONAL BUSINESS MACHINES CORPORATION, Armonk, NY (US)

(72) Inventors: Jean Wynne, Nassau, NY (US); Marc A. Bergendahl, Rensselaer, NY (US); Jonathan Fry, Fishkill, NY (US); Christopher Waskiewicz, Rexford, NY (US); Christopher J. Penny, Saratoga Springs, NY (US); James J. Demarest, Rensselaer, NY (US)

(21) Appl. No.: 15/830,238

(22) Filed: Dec. 4, 2017

(51) Int. Cl. A61B 7/00; G16H 20/60; A61B 5/01; A61B 5/00; A61B 5/11; A61F 13/42

(52) U.S. Cl. CPC A61B 7/008; G16H 20/60; A61B 5/01; A61B 5/4878; A61B 5/0077; A61B 2503/04; A61F 13/42; A61B 5/7267; A61B 5/746; A61B 5/0004; A61B 5/1123

(57) ABSTRACT

An infant gastrointestinal monitor and a method for infant gastrointestinal monitoring are provided. The infant gastrointestinal monitor includes a belly band for placing around at least a midsection area of a subject infant. The infant gastrointestinal monitor further includes a plurality of wireless sound sensors, integrated with the belly band in an array configuration, for identifying a location of a gastrointestinal noise in the subject infant based on cross-referencing signals from the plurality of wireless sound sensors. He infant gastrointestinal monitor also includes a controller, operatively coupled to the plurality of wireless sound sensors, for analyzing the location and one or more other parameters of the gastrointestinal noise to identify a probable cause of the noise and a recommended action for a caregiver to alleviate an underlying condition causing the noise.

### BACKGROUND

### Technical Field

The present invention relates generally to wearable health devices and, in particular, to an infant gastrointestinal monitor.

### Description of the Related Art

When a baby cries, it is difficult to figure out why, which in turn causes stress for the care taker. Many times the cause is related to some type of gastrointestinal distress including, for example, hunger, a need to burp, and a need to pass gas.

Managing time as a caregiver is difficult, and it would be valuable to understand when care is likely to be needed in order to, for example, prepare for the type of care to be administered (e.g., feeding, diaper change), plan other chores errands around the care (e.g., shopping, laundry, nap, etc.), and know what supplies to have on hand. Hence, there is a need for an infant baby monitor capable of providing enhanced information.

### SUMMARY

According to an aspect of the present invention, an infant gastrointestinal monitor is provided. The infant gastrointestinal monitor includes a belly band for placing around at least a midsection area of a subject infant. The infant gastrointestinal monitor further includes a plurality of wireless sound sensors, integrated with the belly band in an array configuration, for identifying a location of a gastrointestinal noise in the subject infant based on cross-referencing signals from the plurality of wireless sound sensors. He infant gastrointestinal monitor also includes a controller, operatively coupled to the plurality of wireless sound sensors, for analyzing the location and one or more other parameters of the gastrointestinal noise to identify a probable cause of the noise and a recommended action for a caregiver to alleviate an underlying condition causing the noise.

According to another aspect of the present invention, a method is provided for infant gastrointestinal monitoring. The method includes identifying, by a plurality of wireless sound sensors integrated with a belly band in an array configuration and placed around at least a midsection area of a subject infant, a location of a gastrointestinal noise in the subject infant based on cross-referencing signals from the plurality of wireless sound sensors. The method further includes analyzing, by a controller operatively coupled to the plurality of wireless sound sensors, the location and one or more other parameters of the gastrointestinal noise to identify a probable cause of the noise and a recommended action for a caregiver to alleviate an underlying condition causing the noise.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 shows an exemplary processing system to which the present invention may be applied, in accordance with an embodiment of the present invention;

FIG. 2 shows an exemplary base infant gastrointestinal monitor, in accordance with an embodiment of the present invention;

FIG. 3 shows an exemplary enhanced infant gastrointestinal monitor, in accordance with an embodiment of the present invention; and

FIG. 4 shows an exemplary method for use with an infant monitor, in accordance with an embodiment of the present invention.

### DETAILED DESCRIPTION

The present invention is directed to an infant gastrointestinal monitor.

In an embodiment, the present invention can provide one or more of the following: assisted continuous diagnostic logging for future reference; specific highlights for parents and/or physicians; action to outcome tracking without medical/diagnostic input (non-medical system); and a predictive outlook and notification for parents, predictive time queries and planning capabilities.

In an embodiment, the present invention can be configured to focus on routine “everyday” domestic factors of an infant.

In an embodiment, the present invention can involve the application of smart technology with a wireless stethoscope to monitor gastrointestinal noises of an infant's stomach. In an embodiment, multiple sensors imbedded in a belly band worn by an infant can be used to identify the location of noise in the infant. In an embodiment, an analysis of the location and type/frequency of noise can be used to identify a probable cause(s) of discomfort and recommended action(s) for a care taker.

In an embodiment, any action taken is manually recorded, then the infant monitor can determine if the action remediated the stress or if further action is required. In an embodiment, a prioritized list of actions can be provided to a caregiver such as, for example, try action A, then action B, then action C, and based on what worked from the sensor data or caretaker feedback, re-weight the actions the next time this occurs such that, for example, it is suggested to try action B, then action C and action A, if action B is unsuccessful. In an embodiment, the smart technology can use feedback to improve the aforementioned analysis and personalize the recommended action(s) for the infant. In an embodiment, the present invention can provide alarms/alerts and predicative analytics for managing the care of the baby

Some of the many attendant benefits of the present invention include, but are not limited to, for example: reducing the time needed to identify the cause of an infant's stress; improving the success rate of the first action implemented to address the infant's stress; reducing the stress of the care taker; and so forth. Moreover, the present invention can be used to gather information on Sudden Infant Death Syndrome (SIDS) and other poorly understood infant conditions

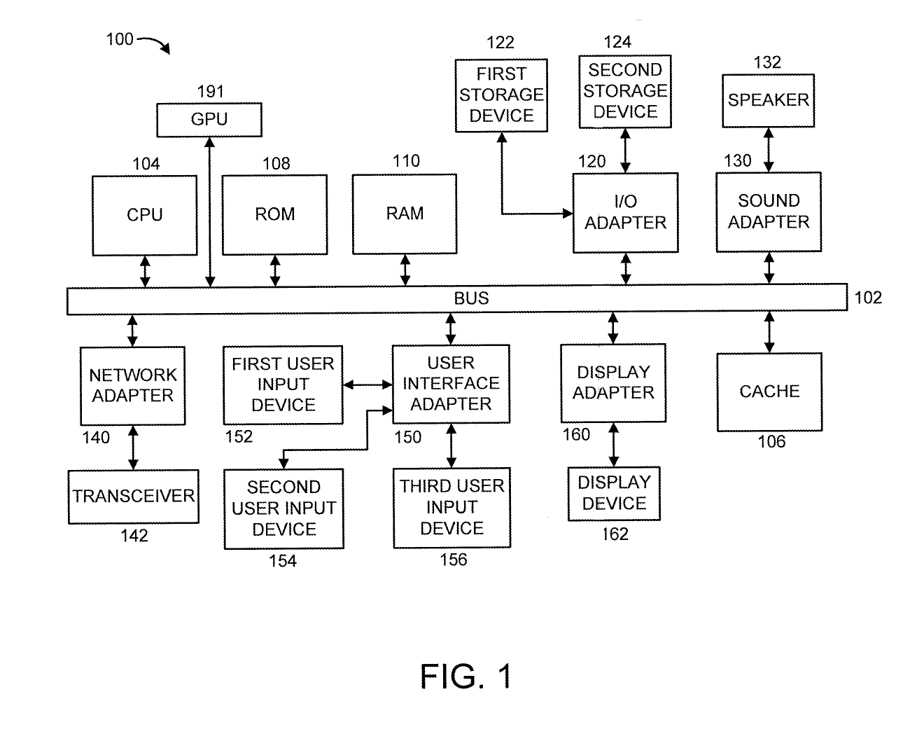

FIG. 1 shows an exemplary processing system 100 to which the invention principles may be applied, in accordance with an embodiment of the present invention. The processing system 100 includes at least one processor (CPU) 104 operatively coupled to other components via a system bus 102. A cache 106, a Read Only Memory (ROM) 108, a Random Access Memory (RAM) 110, an input/output (I/O) adapter 120, a sound adapter 130, a network adapter 140, a user interface adapter 150, and a display adapter 160, are operatively coupled to the system bus 102. At least one Graphics Processing Unit (GPU) 191 is operatively coupled to the system bus 102.

A first storage device 122 and a second storage device 124 are operatively coupled to system bus 102 by the I/O adapter 120. The storage devices 122 and 124 can be any of a disk storage device (e.g., a magnetic or optical disk storage device), a solid state magnetic device, and so forth. The storage devices 122 and 124 can be the same type of storage device or different types of storage devices.

A speaker 132 is operatively coupled to system bus 102 by the sound adapter 130. A transceiver 142 is operatively coupled to system bus 102 by network adapter 140. A display device 162 is operatively coupled to system bus 102 by display adapter 160.

A first user input device 152, a second user input device 154, and a third user input device 156 are operatively coupled to system bus 102 by user interface adapter 150. The user input devices 152, 154, and 156 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 152, 154, and 156 can be the same type of user input device or different types of user input devices. The user input devices 152, 154, and 156 are used to input and output information to and from system 100.

Of course, the processing system 100 may also include other elements (not shown), as readily contemplated by one of skill in the art, as well as omit certain elements. For example, various other input devices and/or output devices can be included in processing system 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 100 are readily contemplated by one of ordinary skill in the art given the teachings of the present invention provided herein.

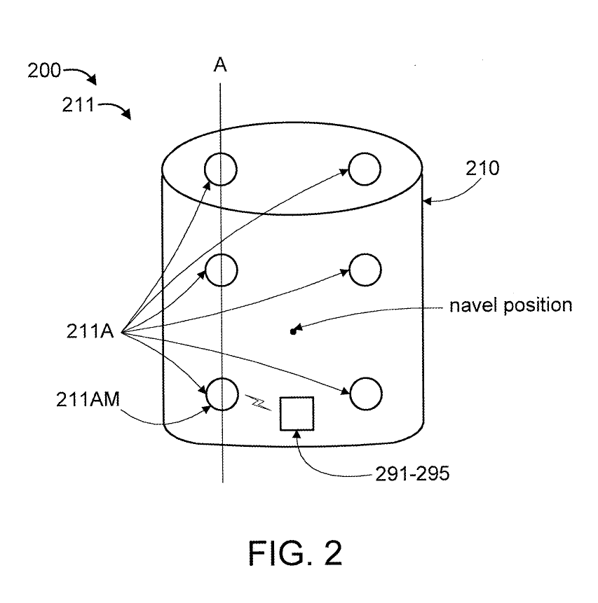

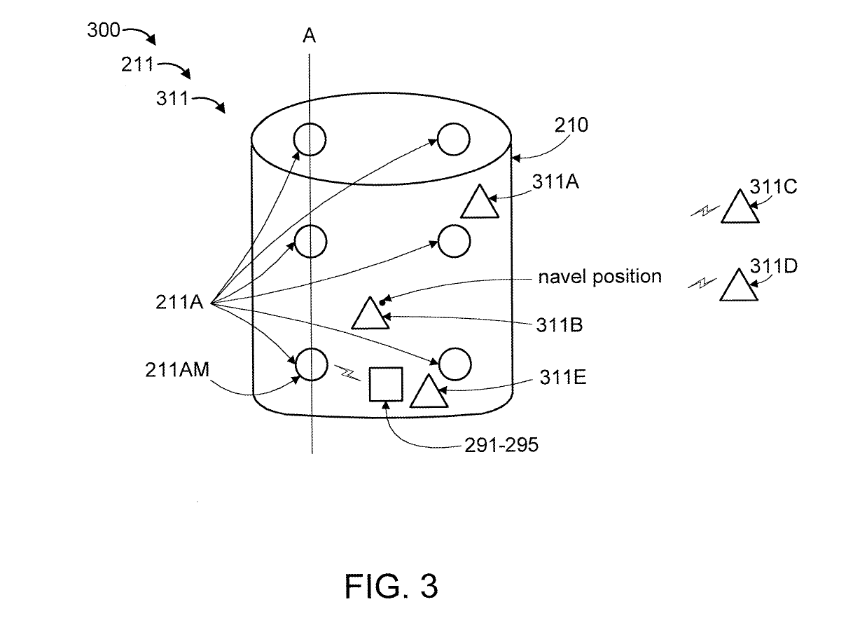

It is to be appreciated that a system such as processing system 100 may be used to interface with an infant monitor such as the baby bands 210 and 310 shown and described with respect to FIGS. 2 and 3, respectively. The baby bands 210 and 310 are interchangeably referred to herein as “infant monitors” and “infant gastrointestinal monitors”. While a particular processing system 100 is shown and described with respect to FIG. 1 for the sake of illustration, it is to be appreciated that the present invention can be applied to and interface with other types of devices including, but not limited to, smart phones, media playback devices, laptops, tablets, smart watches, and so forth.

FIG. 2 shows an exemplary base infant gastrointestinal monitor 200, in accordance with an embodiment of the present invention.

The infant gastrointestinal monitor 200 can include a wireless stethoscope 211 integrated with a belly band 210. The wireless stethoscope 211 can include multiple wireless stethoscope sensors 211A (e.g., eight for the sake of illustration, although only six can be seen in the view of FIG. 2, with the top 2 pertaining to the back of the belly band 210 and the bottom four pertaining to the front of the belly band 210) integrated with the belly band 210. The wireless stethoscope sensors 211A are shown as circles in FIGS. 2 and 3. The sensors 211A can be configured to capture audible and inaudible sounds/frequencies. The sensors 211A can be used to detect conditions including, but not limited to, for example: reflux; gas; diarrhea; and so forth. The type and location of sounds (e.g., based on multi-sensor consensus), aids in diagnosing the condition. For example, heartbeat and heartrate are both indicators of stress. Many different types of sounds can be detected including, but not limited to, breathing or irregular breathing, coughing, wheezing, crying, happy noises, and so forth. In an embodiment, external sounds can be removed from consideration using enhanced signal filtering (e.g., external noise cancellation). In another embodiment, external sounds can be considered to determine if they are a possible cause of distress (e.g., barking dog, fire alarm, and so forth).

In an embodiment, one of the sensors 211A can be configured as a master sensor 211AM to collect and send the data from the wireless stethoscope 211 to a wireless transceiver 293 (described hereinafter) or to an external device (e.g., computer processing system 100). As used herein, “integrated with” can involve, but is not limited to, any of the following: put in; put on; made as part of; connected to; adhered to; and so forth. For example, in an embodiment, the eight wireless stethoscope sensors 211A are embedded in the belly band 210. The belly band 210 can be formed from one or more materials, at least one of which having elastic properties (e.g., but not limited to Lycra, Spandex, and so forth). The belly band 210 is configured to be worn around an infant's mid region. Four sensors 211A are located on the front of the belly band 210 and four sensors 211A are located on the back of the belly band 210.

In an embodiment, the sensors 211A are arranged in an array. For example, in an embodiment, the belly band 210 is to be placed on (around) an infant such that two of the front sensors 211A and two of the back sensors 211A are above the navel. When worn properly, the remaining four sensors 211A are below the navel of the infant. Also, when worn properly, two of the front sensors 211A and two of the back sensors 211A are to the left of the navel and the remaining four sensors 211A are to the right of the navel.

The belly band 210 can be offered in various sizes to fit infants of various sizes/ages. The sensors 211A are fixed in the belly band 210 such that they rest on the line half way between the infant's navel and left or right side (represented as line A in FIG. 2), and the same distance above or below the navel.

In an embodiment, the wireless stethoscope 211 can be configured to collect data and transmit the data to an external device (e.g., computer processing system 100). In another embodiment, a controller 291, associated memory 292, and a wireless transceiver 293 can be used to receive data from the wireless stethoscope 211 and then transmit the data to the external device. In an embodiment, the controller 291 can include and/or otherwise be coupled to a display 294 and/or a speaker 295. An output of the infant gastrointestinal monitor could be output on the display 294 and/or the speaker 295 and/or on the external device. The elements 291-295 are shown as a square in FIGS. 2 and 3. While a single square is used, the elements may be separate or at least some combined as mentioned above.

In an embodiment, the controller 291 is configured to use any provided caregiver feedback in order to identify the probable cause of discomfort and the recommended action for the caregiver to alleviate the discomfort. In an embodiment, the controller 291 is configured to use predictive analysis along with the feedback in order to provide a predictive schedule for the caregiver relative to care to be given to the subject infant. In an embodiment, the controller 291 is configured to learn from a response by the subject infant to the recommended action to improve future recommended actions. Hence, for example, the present invention can change from one recommended action to another for the same or two separate occurrences of the same problem, when a first recommended action does not have the intended curative affect.

In an embodiment, one or more devices can be integrated together. For example, in an embodiment, at least the controller 291, memory 292, and wireless transceiver 293 are formed in a single device. For example, an Application Specific Integrated Circuit (ASIC) and so forth can be used to implement one or more of these elements. These and other configurations and variations are readily determined by one of ordinary skill in the art given the teachings of the present invention provided herein, while maintaining the spirit of the present invention.

FIG. 3 shows an exemplary enhanced infant gastrointestinal monitor 300, in accordance with an embodiment of the present invention.

The enhanced infant gastrointestinal monitor 300 can include the wireless stethoscope 211 with eight stethoscope sensors from the base belly band 200, as well as additional sensors/devices 311. The additional sensors 311 can be integrated with the belly band 210 similar to the eight stethoscope sensors or can be operatively coupled to (e.g., in signal communication with) one or more elements of the infant gastrointestinal monitor (e.g., the controller 291, memory 292, and/or transceiver 293) or can be operatively coupled to (e.g., in signal communication with) an external device (e.g., computer processing system 100). In the latter case, a same software application on the external device can be used to interface with all of the sensor data by a caregiver. Hence, the additional sensors 311 can be integrated with the belly band 210 or be proximate to the belly band 210 in order to supplement the wireless stethoscope data. The additional sensors 311 are shown as triangles in FIGS. 2 and 3.

In an embodiment, the additional sensors 311 can include, but are not limited to, for example: a skin surface temperature sensor 311A; a water retention/bloat sensor 310B; an infrared video capture device 311C for measuring temperature and body position; a video capture device 311D for detecting rooting behavior indicating hunger as well as physical dimension variations (e.g., swelling, bloat, etc.); and a diaper condition (e.g., moisture) sensor 311E for determining a diaper condition (e.g., soiled, not soiled). In an embodiment, the additional sensors 311 can be integrated with the belly band 210 and/or placed proximate to the belly band 210 to provide supplemental sensor data. In an embodiment, the water retention/bloat sensor 310B could be implemented by either a skin surface band (e.g., involving an elastic measurement) or using an intake-outflow type comparison, selectively coupled with the diaper sensor output and caregiver actions (to allow data entry for intake and/or outflow).

In an embodiment, each of the additional sensors 311 is wireless, to facilitate ease of communication. Of course, other connection arrangements (e.g., hardwired) can also be used, while maintaining the spirit of the present invention.

Of course, other types of sensors can also be used, as readily contemplated by one of ordinary skill in the art, given the teachings of the present invention provided herein, while maintaining the spirit of the present invention.

In an embodiment, input parameters can include, but are not limited to, for example: (I1) location of stomach noise; (I2) wave length of stomach noise; (I3) frequency of stomach noise; (I4) action taken by care taker; (I5) past history/log (O2); (I6) diaper condition sensor. Sources for the input parameters can include, but are not limited to, for example: wireless stethoscopes; manual data input; and so forth. Methods including, but not limited to, spectral analysis (e.g., Short Time Fourier Transform (STFT) or Wavelet Channel Energy/Wavelet Decomposition) can be used to parse audio signals for further analysis rather than just wavelength/frequency analysis.

In an embodiment, the following evaluation/action selection process can be performed by an infant gastrointestinal monitor in accordance with the present invention: (1) an evaluation of the location of stomach noise in the digestive tract; and (2) a characterization of the stomach noise wave length and frequency. In an embodiment, the detected gastrointestinal noises can be compared to a database of gastrointestinal sound parameters (e.g., locations, frequencies, wavelength, etc.) and corresponding curative actions (e.g., stored in memory 292) in order to perform comparisons to detect a particular source of a particular gastrointestinal sound and a curative action(s) corresponding thereto. Moreover, as mentioned above, the present invention can learn from past experiences and save the most effective action(s) for a given gastrointestinal noise. To that end, any machine learning technique and/or mechanism can be used including, but not limited to, for example, decision trees, rule-based learning, deep learning, neural networks, support vector machines, clustering, Bayesian networks, and so forth. It is to be appreciated that the preceding are merely illustrative and, thus, other machine learning techniques and/or mechanisms can also be used in accordance with the teachings of the present invention, while maintaining the spirit of the present invention. Caregiver feedback can be used to train/retrain any learning mechanism used in order to provide the optimum actions for a given condition. Training data can also be derived from medical journals and other sources.

In an embodiment, an output of the present invention can include, but is not limited to, for example: (O1) a recommended action(s) for a care taker; and (O2) a medical/data log history update. In an embodiment, the field performance data of a plurality of these infant monitor devices can be aggregated to push a firmware/learn database update to the infant monitor, based on actual performance.

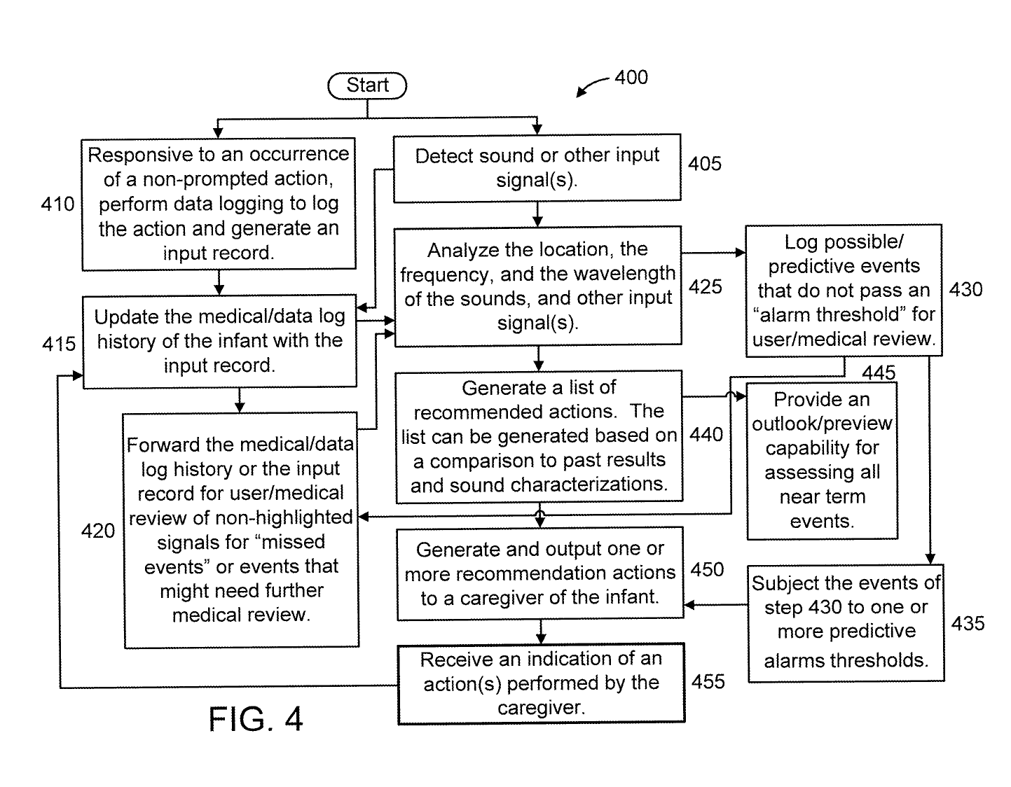

FIG. 4 shows an exemplary method 400 for use with an infant gastrointestinal monitor, in accordance with an embodiment of the present invention. The method 400 presupposes that a caregiver has placed the infant gastrointestinal monitor on the infant. The infant gastrointestinal monitor can be configured similar to the base infant gastrointestinal monitor 200 shown and described with respect to FIG. 2 or the enhanced infant gastrointestinal monitor 300 shown and described with respect to FIG. 3.

At step 405, detect sound or other input signal(s). The other input signal(s) can include, but is(are) not limited to, for example: a skin temperature input signal; a moisture input signal; an infrared video input signal; and a video input signal.

At step 410, responsive to an occurrence of a non-prompted action, perform data logging to log the action and generate an input record. The non-prompted action can include, but is not limited to, for example, regular feeding, diaper/clothes changes, burping, and so forth.

At step 415, update the medical/data log history of the infant with the input record.

At step 420, forward the medical/data log history or the input record for user/medical review of non-highlighted signals for “missed events” or events that might need further medical review. External recommendations can be captured for updates to the method 400.

At step 425, analyze the location, the frequency, and the wavelength of the sounds, and other input signal(s).

At step 430, log possible/predictive events that do not pass an “alarm threshold” for user/medical review.

At step 435, subject the events of step 430 to one or more predictive alarms thresholds. In an embodiment, step 435 can be performed depending upon the implementation.

At step 440, generate a list of recommended actions. In an embodiment, the list can be generated based on a comparison to past results and sound characterizations.

At step 445, provide an outlook/preview capability for assessing all near term events. For example, provide a capability for the user to pose queries and receive responses from the infant gastrointestinal monitor. Such a query can be, but is not limited to, for example: “Can I start cooking or do I need to take action first to prevent interruption?”. Other example queries can be as follows: “What methods should I use to comfort/address the baby's issue, and which ones work for my baby?”; “What do I need to schedule around?”; “How much free time do I have?”; “Can I fit X into the schedule?”; and “When I start preparing for the next event, what should I prepare in advance?”.

At step 450, generate and output one or more recommendation actions to a caregiver of the infant.

At step 455, receive an indication of an action(s) performed by the caregiver. The action(s) can be a recommended or non-recommended (i.e., any) action(s). The action(s) can be entered manually by a user or detected by the system. In this way, a non-recommended action can be evaluated by the system for possible suggestion upon a re-occurrence of the underlying condition.

A description will now be given regarding an embodiment for using an infant gastrointestinal monitor, in accordance with an embodiment of the present invention.

An initial use of the infant gastrointestinal monitor can rely on input only and general medical knowledge programing to provide advice to caregiver about current or imminent condition. For example, regarding general medical knowledge programming, a Bayesian baseline model or other type of model can be used.

For example, lower abdomen noises typically indicate that gas release is needed. Accordingly, an example output from the infant gastrointestinal monitor can be as follows: “Move baby's legs to help release gas.”

Beginning with a first use, the present invention can collect user input and log outcomes specific to the infant being monitored to increase the predictive knowledge. For example, in an embodiment, the present invention can prompt you through walking, tapping, and rocking as different methods/actions. Moreover, in an embodiment, the present invention can note and/or otherwise highlight the fact that tapping appears to be most effective based on manual feedback and sensor confirmation.

A description will now be given regarding another embodiment for using an infant gastrointestinal monitor, in accordance with an embodiment of the present invention.

Predictive features can be offered immediately or once a chosen confidence interval is reached. As more data is collected, prediction accuracy will improve.

Further to the preceding, consider the following two illustrative examples.

In the first example, the present invention provides an anticipatory alarm capability (time-based) and user selectable thresholds for event notification and prediction. Hence, regarding a predictive trigger for feeding need, a user can choose a lead time (e.g., 20 minutes) to the predicated event, or the present invention can suggest a lead time based on the required preparation (e.g., 10 minutes to prepare a bottle).

In the case when a prediction indicates feeding will likely be at 04:00, the present invention can check user (e.g., caregiver) preferences regarding when to notify the user and can produce a user notification at 03:45.

In the second example, the present invention provides a predictive/near-term outlook. A user can review an upcoming/predictive event to help schedule non-care related activities.

For example, a caregiver queries the present invention for an available time until the next event, and the present invention responds with time lengths and confidence intervals (e.g., 75% chance of 1 hour with no interruptions, 95% chance of 30 minutes with no interruptions).

A prediction schedule can be made available for a caregiver(s) to review that describes the time to the next event. For example, the prediction schedule can predict the next diaper change to be at 3 pm, such that the present invention can determine that the user has 20 minutes to call a neighbor before the next diaper change.

A description will now be given regarding yet another embodiment for using an infant gastrointestinal monitor, in accordance with an embodiment of the present invention.

The embodiment relates to travel preparation. A user loads travel plans or errand lists, to calculate an anticipated time out of the house.

The present invention can suggest necessary supplies for anticipated gastric events while out (e.g., maybe you do not need the diaper bag, feeding bag, and third change of clothes) or alternatively suggests extra supplies based on detected discomfort/activity.

A description will now be given regarding still another embodiment for using a baby monitor, in accordance with an embodiment of the present invention.

The embodiment relates to a logging/non-isolated trending capability. The present invention can highlight items that may have been individually addressed but when reviewed in total indicate a possible larger concern.

For example, the present invention may note that for X out of Y bowel cycles in the last month, the periodicity of movement changed in a similar way for 1 event (3 hour delay to normal). In such a case, the present invention can, for example, perform one of more of the following: notify a caregiver(s) of potential pattern to review and call attention to a possible issue; suggest to the caregiver(s) to review food intake around those noted events and around a next potential event; and makes note in a medical log for a physician/professional review.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

1. An infant gastrointestinal monitor, comprising:
a belly band, having only two openings, for placing around at least a midsection area of a subject infant;
a plurality of wireless sound sensors, integrated with the belly band in at least a vertical array configuration, wherein the belly band is adapted to have at least one of the plurality of wireless sound sensors in an above-the navel position on the belly band relative to the subject infant and at least another one of the plurality of sensors in a below-the navel position on the belly band relative to the subject infant; and
a controller, operatively coupled to the plurality of wireless sound sensors, cross-referencing signals from the plurality of wireless sound sensors to identify a location of a gastrointestinal noise in the subject infant and analyzing the location and one or more other parameters of the gastrointestinal noise to identify a probable cause of the noise and a recommended action for a caregiver to alleviate an underlying condition causing the noise.

2. The infant gastrointestinal monitor of claim 1, wherein the one or more other parameters of the gastrointestinal noise comprise a noise type and a frequency of the gastrointestinal noise.

3. The infant gastrointestinal monitor of claim 1, wherein the plurality of wireless sound sensors comprises at least two audible sound sensors.

4. The infant gastrointestinal monitor of claim 1, further comprising a transceiver for transmitting infant related data to a remote device.

5. The infant gastrointestinal monitor of claim 1, further comprising a skin surface temperature sensor, operatively coupled to the controller, for sensing a skin surface temperature of the subject infant.

6. The infant gastrointestinal monitor of claim 1, further comprising a water monitor, operatively coupled to the controller, for detecting water retention and bloat in the subject infant.

7. The infant gastrointestinal monitor of claim 1, further comprising an infrared video capture device, operatively coupled to the controller, for measuring a body temperature and determining a body position of the subject infant.

8. The infant gastrointestinal monitor of claim 1, further comprising a video capture device, operatively coupled to the controller, for detecting (i) rooting behavior indicative of hunger and (ii) physical dimension variations in the subject infant.

9. The infant gastrointestinal monitor of claim 1, further comprising a moisture sensor, operatively coupled to the controller, for detecting a diaper condition of a diaper worn by the subject infant.

10. The infant gastrointestinal monitor of claim 1, further comprising a display device for displaying the probable cause of discomfort and the recommended action for the caregiver to alleviate the discomfort.

11. The infant gastrointestinal monitor of claim 1, wherein the controller is configured to use a provided caregiver feedback in order to identify the probable cause of discomfort and the recommended action for the caregiver to alleviate the discomfort.

12. The infant gastrointestinal monitor of claim 1, wherein the controller is configured to use predictive analysis along with the feedback in order to provide a predictive schedule for the caregiver relative to care to be given to the subject infant.

13. The infant gastrointestinal monitor of claim 1, wherein the controller is configured to learn from responses by the subject infant and other subject infants, the caregiver, medical personnel, and manually entered data relative to the recommended action to improve future recommended actions.

14. The infant gastrointestinal monitor of claim 1, further comprising a speaker for providing an audible alert to a caregiver responsive to a detection of a particular condition by the plurality of wireless sound sensors.

15. The infant gastrointestinal monitor of claim 1, wherein the controller is configured to determine whether further action is needed and to recommend the further action, based on an item selected from the group consisting of a response by the subject infant to the recommended action and feedback from the caregiver as to an efficacy of the recommended action.

16. The infant gastrointestinal monitor of claim 1, wherein the memory stores at least a portion of a care history of the subject infant, and wherein the controller consults the portion of the care history in order to identify the underlying condition causing the noise and the recommended action for the caregiver to alleviate the underlying condition causing the noise.

17. The infant gastrointestinal monitor of claim 16, wherein the recommendation action is tailored to the subject infant based on at least the portion of the medical history of the subject infant and data from the plurality of wireless sound sensors.

18. The infant gastrointestinal monitor of claim 1, wherein the controller analyzes the location and the one or more other parameters of the gastrointestinal noise using a spectral analysis technique.

19. A method for infant gastrointestinal monitoring, comprising:
identifying, by a controller operatively coupled to a plurality of wireless sound sensors integrated with a belly band placed around at least a midsection area of a subject infant in at least a vertical array configuration, wherein the belly band is adapted to have at least one of the plurality of wireless sound sensors in an above-the navel position on the belly band relative to the subject infant and at least another one of the plurality of sensors in a below-the navel position on the belly band relative to the subject infant, a location of a gastrointestinal noise in the subject infant based on cross-referencing signals from the plurality of wireless sound sensors, the belly band having only two openings; and
analyzing, by the controller, the location and one or more other parameters of the gastrointestinal noise to identify a probable cause of the noise and a recommended action for a caregiver to alleviate an underlying condition causing the noise.

20. The method of claim 19, wherein the one or more other parameters of the gastrointestinal noise comprise a noise type and a frequency of the gastrointestinal noise.
